(19) United States
(12) Patent Application Publication
(10) Pub. No.: US 2024/0317923 A1
(43) Pub. Date: Sep. 26, 2024

(54) A Hemostatic Tissue Adhesive Composition Comprising An NCO-Terminated Urethane Preprolymer

(71) Applicants: Peters Surgical, Boulogne-Billancourt (FR); Centre National de la Recherche Scientifique (CNRS), Paris (FR); Universite De Strasbourg, Strasbourg (FR)

(72) Inventors: Valerie Vidal, Carqueiranne (FR); Sophie Wendels, Strasbourg (FR); Luc Averous, Wiwersheim (FR)

(73) Assignees: Peters Surgical, Boulogne-Billancourt (FR); Centre National de la Recherche Scientifique (CNRS), Paris (FR); Universite De Strasbourg, Strasbourg (FR)

(21) Appl. No.: 18/268,830

(22) PCT Filed: Dec. 22, 2021
(86) PCT No.: PCT/EP2021/087366

§ 371 Date: Jun. 21, 2023

(30) Foreign Application Priority Data
Dec. 23, 2020 (EP) 20306682.4

(51) Int. Cl. C08G 18/42; A61L 26/00; C07C 67/02; C08G 18/10; C08G 18/73; C08G 18/75; C08G 63/06

(52) U.S. Cl. CPC C08G 18/4283; A61L 26/0019; C07C 67/02; C08G 18/10; C08G 18/73; C08G 18/757; C08G 63/06

(57) ABSTRACT

The invention relates to a NCO-terminated urethane preprolymer based on at least a polyol oligomer and a diisocyanate, wherein the polyol oligomer is polyhydroxyalkanoate-diol oligomers (oligoPHA-diol) having a hydroxyl value of more than or equal to 149 mgKOH/g, and the diisocyanate is selected from the list consisting of 1,4-butane diisocyanate (BDI), hexamethylene diisocyanate (6-HDI), dimeryl diisocyanate (DDI), pentamethylene diisocyanate (PDI), L-lysine diisocyanate (LDI), 1,7-heptamethylene diisocyanate (7-HDI), and mixtures thereof. The invention also relates to a method for preparing such a NCO-terminated urethane prepolymer and for a method for preparing polyhydroxyalkanoate-diol oligomers (oligoPHA-diol). The invention also relates to a hemostatic tissue adhesive composition and a hemostatic tissue adhesive. The invention also relates to a kit for the preparation of a hemostatic tissue adhesive.

### FIELD OF THE INVENTION

The invention relates to a hemostatic tissue adhesive composition comprising A) an NCO-terminated urethane preprolymer, and B) a chain extender. The invention also relates to the NCO-terminated urethane preprolymer as such and to the method for its preparation. The invention also relates to a hemostatic tissue adhesive and a kit for preparing the hemostatic tissue adhesive.

### TECHNICAL BACKGROUND

In the last decades, biodegradable tissue adhesives have gained a lot of attention in the medical field, mainly due to increasing surgical procedures around the world and their non-invasiveness compared to golden-standard sutures.

Surgical adhesives are divided into three main categories: (i) hemostatic agents, (ii) Glues, and (iii) sealants. (i) Hemostatic agents acting for bleeding control, usually comprise blood components such as fibrin, thrombin or coagulation factor(s) that will increase the coagulation cascade kinetic. Hemostatic agents however offer poor mechanical resistance and adhesion on tissues, coupled with blood disease-transmission risks. (ii) Glues are the second category of tissue adhesives and help strongly attach tissues together or are used in complement of sutures to improve their mechanical resistance. In this class, cyanoacrylate-based glues are well-known for their quick curing time and strong adhesion on various types of tissues. However, along with induced toxicity of their degradation products, cyanoacrylate-based final films usually display brittleness and lack flexibility. Finally, (iii) sealants, also called “hemostatic tissue adhesive” lie in-between hemostatic agents and glues, offering hemostasis coupled with mid-range adhesion and mechanical resistance. They are mostly used to create a physical barrier against blood, other corporal fluids or gas leaks. Surgical sealants and glues mainly exist in forms of liquids that solidify by curing once in contact with the tissue, or gels that absorb fluids and expand while adhering to the targeted tissue, while various types of hemostatic agents are available, such as gels, powders or dressings.

Among the different bioadhesives requirements, biocompatibility, easy handling, fast curing along with limited exothermic reaction and adaptable mechanical properties are keys to a suitable material. Up until now, no ideal solution has been found that answer all these criteria.

Polyurethane-based (PU) adhesives have recently emerged as promising candidate in this matter thanks to their tunable physico-chemical, mechanical and biodegradation properties. Besides, formulation possibilities include the uses of biobased building blocks and their derivatives. The PU adhesion to tissues usually lies in the presence of free —NCO end-group that react with water and surrounding functions in the tissues such as hydroxyls or amino acids, for instance, to give the final polymer.

In this regard, Su et al. [Q. Su, D. Wei, W. Dai, Y. Zhang, Z. Xia, Designing a castor oil-based polyurethane as bioadhesive, Colloids Surf B Biointerfaces 181 (2019) 740-748] recently designed a bioadhesive by incorporation of up to 12 wt % castor oil in poly(ethylene glycol) 400 g·mol⁻¹ (PEG400) and isophorone diisocyanate (IPDI) PU with good adhesion strength and low curing times. Three- and multi-branched NCO-terminated PU have also been synthesized with intermediates adhesion of 20-80 kPa, which lies between fibrin (2-18 kPa) and cyanoacrylate (650 kPa) [A. I. Bochyiska, S. Sharifi, T. G. van Tienen, P. Buma, D. W. Grijpma, Development of Tissue Adhesives Based on Amphiphilic Isocyanate-Terminated Trimethylene Carbonate Block Copolymers, Macromolecular Symposia 334(1) (2013) 40-48, and A. I. Bochyiska, T. G. Van Tienen, G. Hannink, P. Buma, D. W. Grijpma, Development of biodegradable hyper-branched tissue adhesives for the repair of meniscus tears, Acta Biomater 32 (2016) 1-9]. Xylose has also been successfully incorporated up to 15 polyol mol % in PEG200/4,4′-Methylenebis(cyclohexyl isocyanate) (HMDI) PU structures [S. Balcioglu, H. Parlakpinar, N. Vardi, E. B. Denkbas, M. G. Karaaslan, S. Gulgen, E. Taslidere, S. Koytepe, B. Ates, Design of Xylose-Based Semisynthetic Polyurethane Tissue Adhesives with Enhanced Bioactivity Properties, ACS Appl Mater Interfaces 8(7) (2016) 4456-66].

Nonetheless, excessive swelling remains a threat to mechanical properties and adhesion in some crosslinked network. To the best of our knowledge and considering “biobased” a material comprising at least 25 wt % biobased components, only a few biobased PU bioadhesives have been reported until now.

Furthermore, fast curing time is an essential property for surgical adhesives, as a too low curing time can lead to fluids and/or blood leaks and may further cause severe damage to the patient. Most isocyanate-based adhesive have shown long curing time, usually several hours are needed for a complete NCO disappearance.

There is thus a need for designing new biobased NCO-terminated PU prepolymers as tissue adhesives with tunable adhesion, mechanical and biodegradation properties that could be suitable for various kinds of surgeries. There is also a need for designing NCO-terminated PU prepolymers based surgical adhesives with fast curing time.

### SUMMARY OF THE INVENTION

Polyhydroxyalkanoates (PHAs) are a biodegradable and biobased family of bacterial polyesters with different architectures. Among them, the poly-3-hydroxybutyrate (PHB) is the most conventional and easily obtained by biotechnologies from different bioresources. However, this thermoplastic polyester known since more than 50 years presents until now only very limited applications due to a certain number of drawbacks such as poor mechanical properties, high degree of crystallinity, and low thermal stability. One major way to valorize this bacterial polyester could be to develop controlled building blocks for the synthesis of new generation of biobased polymers.

In this way, the inventors have successfully prepared short linear PHA-diols oligomers (oligoPHA-diol), including short linear PHB-diols oligomers (oligoPHB-diol), with controlled low molar masses from a new synthetic pathway using reactive solvents in a green way.

Then, the inventors have successfully prepared thermoplastic polyurethanes (TPU) with high biobased content and have thus successfully designed new biobased NCO-terminated PU prepolymers as tissue adhesives with tunable adhesion, mechanical and biodegradation properties that could be suitable for various kinds of surgeries.

In a first aspect, the invention relates to a NCO-terminated urethane preprolymer based on at least a polyol oligomer and a diisocyanate, wherein the polyol oligomer is polyhydroxyalkanoate-diol oligomer (oligoPHA-diol) having a hydroxyl value of more than or equal to 149 mgKOH/g, and the diisocyanate is selected from the list consisting of 1,4-butane diisocyanate (BDI), hexamethylene diisocyanate (6-HDI), dimeryl diisocyanate (DDI), pentamethylene diisocyanate (PDI), L-lysine diisocyanate (LDI), 1,7-heptamethylene diisocyanate (7-HDI), and mixtures thereof.

In a second aspect, the invention relates to a method for preparing polyhydroxyalkanoate-diol oligomers (oligoPHA-diol) having a hydroxyl value of more than or equal to 149 mgKOH/g, comprising the following steps:
a) Heating a reactive solvent at a temperature of more than or equal to the melting point of the PHA and below the reactive solvent boiling point, in particular of between 170° C. and 190° C., advantageously of 180° C., under inert gaz flow, the reactive solvent being selected in the group consisting of 1,2 ethylene glycol, 1,3-propanediol, 1,4-butanediol, and mixtures thereof, preferably 1,4-butanediol;
b) Adding dried PHA and stirring;
c) Starting the reaction by adding a catalyst;
d) Allowing to react for a period of between 15 and 240 minutes, advantageously between 100 and 215 minutes, typically between 205 and 215 minutes;
e) Precipitating the mixture obtained after step d) and washing; and
f) Recovering the oligoPHA-diol by distillation at a temperature of between 120° C. and 180° C., advantageously between 150° C. and 180° C., typically between 140° C. and 160° C., under reduced pressure.

In a third aspect, the invention relates to a method for preparing the NCO-terminated urethane prepolymer of the invention, comprising the following steps:
a′) Mixing together the diisocyanate with the polyhydroxyalkanoate-diol oligomer (oligoPHA-diol) at a temperature of between 20° C. and 110° C., under stirring, the diisocyanate being selected from the list consisting of 1,4-butanediisocyanate (BDI), hexamethylene diisocyanate (6-HDI), dimeryl diisocyanate (DDI), pentamethylene diisocyanate (PDI), L-lysine diisocyanate (LDI), 1,7-heptamethylene diisocyanate (7-HDI), and mixtures thereof, and the oligoPHA-diol having a hydroxyl value of more than or equal to 149 mgKOH/g and/or being obtained according to the method of the second aspect of the invention;
b′) Optionally, when a mixture of DDI with a second diisocyanate is used, adding in step a′) the DDI and in step b′) adding dropwise the second diisocyanate being selected from the list consisting of 1,4-butanediisocyanate (BDI), pentamethylene diisocyanate (PDI), L-lysine diisocyanate (LDI), hexamethylene diisocyanate (6-HDI), 1,7-heptamethylene diisocyanate (7-HDI), and mixtures thereof;
c′) Allowing to react until obtaining a free —NCO content of between 4 and 18% by weight, in relation to the total weight of the prepolymer, measured by determining the free —NCO content by an indirect-titration method.
d′) Recovering the NCO-terminated urethane prepolymer.

In a fourth aspect, the invention relates to a hemostatic tissue adhesive composition comprising A) the NCO-terminated urethane preprolymer of the invention or as obtained according to the method of the invention, and B) a chain extender.

In a fifth aspect, the invention relates to a hemostatic tissue adhesive obtained by reacting A) the NCO-terminated urethane prepolymer of the invention or as obtained by the method of the invention; and B) a chain extender advantageously selected from the group consisting of N-ethyldiethanolamine, N-butyldiethanolamine, 1,4-butanediol, polyethylene glycol, polypropylene glycol, 1-amino-4-butanol, and mixtures thereof, more advantageously 1,4-butanediol.

Another aspect of the invention is a kit for the preparation of a hemostatic tissue adhesive comprising a composition A comprising the NCO-terminated urethane preprolymer of the invention or as obtained according to the method of the invention, and a composition B comprising a chain extender advantageously selected from the group consisting of ethyldiethanolamine, N-butyldiethanolamine, 1,4-butanediol, polyethylene glycol polypropylene glycol, 1-amino-4-butanol, and mixtures thereof, more advantageously 1,4-butanediol, the compositions A and B being packaged separately and being parenterally administrable simultaneously, sequentially or separately.

Another aspect of the invention is the use of the hemostatic tissue adhesive composition of the invention for the preparation of a hemostatic tissue adhesive.

### DETAILED DESCRIPTION OF THE INVENTION

### Definition

In the context of the invention, the term “biobased adhesive” means an adhesive comprising at least 25 wt % biobased components.

In the context of the invention, the term “hemostatic tissue adhesive composition” means a composition based on an NCO-terminated urethane preprolymer and a chain extender, said composition being intended to be applied on a tissue and then to be cured to give a hemostatic tissue adhesive. In the context of the invention the “hemostatic tissue adhesive”, also called “surgical hemostatic adhesive”, is thus obtained by applying the hemostatic tissue adhesive composition to a tissue which is then allowed to cure.

In the context of the invention, the term “polyol oligomer” refers to short PHA polymer chains (oligomers) with hydroxyl end-functions, and with a hydroxyl functionality of more than one. In the context of the invention the hydroxyl functionality of the polyol oligomer is close to 2, advantageously is 2. Thus, in the present application, the terms “polyols”, “polyol oligomer”, and “oligo-polyols” can be used interchangeably, as they refer to the same entity. When the hydroxyl functionality of the polyol oligomer is 2, the terms “diol oligomer”, and “oligoPHA-diol” can be used interchangeably, as they refer to the same entity.

In the context of the invention, the term “prepolymer” means an oligomer or polymer having reactive end-groups that could allow it to participate in a subsequent polymerization by reaction with other reactive functions.

In the context of the invention, the term “NCO-terminated urethane preprolymer”, also called “isocyanate terminated polyurethane prepolymer” is the result of the reaction between one or several diisocyanates and one or several polyols, and in particular between isocyanate groups (—NCO) of the diisocyanate and hydroxyl groups (—OH) of the polyol, in which the obtained prepolymers are —NCO terminated.

In the context of the invention, the term “prepolymer based on” means a prepolymer comprising the mixture of the starting components and/or the product of the reaction between the starting components used for the polymerisation of this prepolymer, preferably only the product of the reaction between the different starting components used for this prepolymer, some of which may be intended to react or may react with each other or with their close chemical environment, at least in part, during the different phases of the process of manufacture of the prepolymer, in particular during a polymerisation stage. Thus, the starting components are the reagents intended to react together during the polymerization of the prepolymer. The starting components are therefore introduced into a reaction mixture optionally additionally comprising a solvent or a mixture of solvents and/or other additives such as at least one catalyser and/or at least one salt and/or at least one polymerization initiator and/or at least one stabilizer.

In the context of the invention, and in particular in the context of oligo-polyols for polyurethanes, the term “hydroxyl value”, also called “hydroxyl index” or “IOH”, defined the quantitative value of the amount of hydroxyl groups available for the reaction with isocyanates. The hydroxyl value is expressed as milligrams of potassium hydroxide equivalent for one gram of the sample (mgKOH/g). The hydroxyl value can be determined by the method as disclosed in the following standards: DIN 53240-2: 2007-11 (2007), ou ASTM E1899-02 (2002).

In the context of the invention, the terms “average molecular weight”, also called “average molar mass” is defined as the average value of the molecular weight distribution profile, where each molecule is considered to contribute equally to the average, also called “number average molecular weight”, or “Mn”. In the invention, the average molar mass of the oligoPHA-diols or prepolymers in solutions can be determined by size exclusion chromatography (SEC), also known as gel permeation chromatography (GPC).

In the context of the invention, the term “reactive solvent” means a molecule added in excess in the reaction media, which is in form of liquid at the reaction temperature and which is acting as solvent but also is actively participating to the targeted reaction.

In the context of the invention, the “NCO/OH molar ratio”, also called “NCO/OH ratio” is defined as the equivalent ratio between the materials containing —NCO groups (diisocyanates) and those containing —OH group (polyols). The NCO/OH molar ratio is an efficient way to regulate the morphology and properties of isocyanate-terminated polyurethane prepolymer. The NCO:OH molar ratio is chosen by determining the hydroxyl index (IOH) (as indicated above) and the NCO index (determined by the method as disclosed in the following standards: DIN 53185, 16945 (1994) or ASTM D1638 (1985).

### NCO-Terminated Urethane Preprolymer

The first aspect of the invention relates to a NCO-terminated urethane preprolymer based on at least a polyol oligomer and a diisocyanate, wherein the polyol oligomer is a polyhydroxyalkanoate-diol oligomer (oligoPHA-diol) having a hydroxyl value of more than or equal to 149 mgKOH/g, and the diisocyanate is selected from the list consisting of 1,4-butane diisocyanate (BDI), hexamethylene diisocyanate (6-HDI), dimeryl diisocyanate (DDI), pentamethylene diisocyanate (PDI), L-lysine diisocyanate (LDI), 1,7-heptamethylene diisocyanate (7-HDI), and mixtures thereof.

Advantageously, the oligoPHA-diol have a hydroxyl value of between 149 mgKOH/g and 560 mgKOH/g, advantageously of between 149 mgKOH/g and 375 mgKOH/g, more advantageously of between 160 mgKOH/g and 375 mgKOH/g, in particular of between 224 mgKOH/g and 375 mgKOH/g.

Advantageously, the oligoPHA-diol have an average molecular weight of less than or equal to 750 g/mol. More advantageously, the oligoPHA-diol have an average molecular weight of between 200 g/mol and 750 g/mol, more advantageously of between 300 g/mol and 750 g/mol, more advantageously of between 300 g/mol and 700 g/mol, and more advantageously of between 300 g/mol and 500 g/mol.

One specific feature of the NCO-terminated urethane preprolymer of the invention is to be obtained from polyol oligomers selected from polyhydroxyalkanoate-diol oligomers (oligoPHA-diol) having a high hydroxyl value, i.e. a hydroxyl value of more than or equal to 149 mgKOH/g, and thus with a low average molecular weight, i.e. an average molecular weight of less than or equal to 750 g/mol. By increasing the oligoPHA-diol hydroxyl value and thus by decreasing the oligoPHA-diol average molecular weight, the physical aspect of the oligomers varies from powdery solids to viscous liquids. Moreover, the oligomers viscosity decreased with the molar mass. The oligoPHA-diols with low viscosities present a great advantage for bulk process, to avoid the use of toxic and environmentally unfriendly organic solvents, with a green chemistry approach. Furthermore, the NCO-terminated urethane preprolymer obtained from oligoPHA-diols with high hydroxyl value and thus low average molecular weight has the advantages of being a viscous liquid with tailored properties.

Advantageously, the oligoPHA-diol are obtained by transesterification of a polyhydroxyalkanoate (PHA) with a reactive solvent selected in the group consisting of 1,2-ethylene glycol, 1,3-propanediol (PDO), 1,4-butanediol (BDO), or mixture thereof. The specific method for obtaining the oligoPHA-diol is disclosed below, in section named “Method for preparing the oligoPHA-diol”.

Advantageously, the PHA is selected from the group consisting of poly-3-hydroxybutyrate (P3HB), poly-4-hydroxybutyrate (P4HB), poly-3-hydroxyvalerate (P3HV), poly-3-hydroxypropionate (P3HP), poly-4-hydroxyvalerate (P4HV), poly-5-hydroxyvalerate (P5HV), and mixtures thereof, advantageously poly-3-hydroxybutyrate (P3HB), poly-4-hydroxybutyrate (P4HB) or mixtures thereof, more advantageously poly-3-hydroxybutyrate (P3HB).

Advantageously, the diisocyanate is a mixture of dimeryl diisocyanate (DDI) with a second diisocyanate selected in the group consisting of 1,4-butanediisocyanate (BDI), pentamethylene diisocyanate (PDI), L-lysine diisocyanate (LDI), hexamethylene diisocyanate (6-HDI), 1,7-heptamethylene diisocyanate (7-HDI), and mixtures thereof. Typically, in such a mixture, the content ratio (BDI, PDI, LDI, 6-HDI, 7-HDI or mixtures thereof)/(DDI) is of between 0/100 and 100/0, advantageously of between 25/75 and 0/100, more advantageously between 50/50 and 0/100, in particular the content ratio (BDI, PDI, LDI, 6-HDI, 7-HDI or mixtures thereof)/(DDI) is 50/50, 25/75, 0,1/99,9 or 0/100. Advantageously, in such a mixture, the content ratio (BDI, PDI, LDI, 6-HDI, 7-HDI or mixtures thereof)/(DDI) is of between 0,1/99,9 and 99,9/0,1, preferably between 20/80 and 80/20 or preferably between 25/75 and 0,1/99,9 or between 50/50 and 0,1/99,9.

More advantageously, the diisocyanate is a mixture of dimeryl diisocyanate (DDI) with a second diisocyanate selected in the group consisting of pentamethylene diisocyanate (PDI), hexamethylene diisocyanate (6-HDI) and mixtures thereof. Typically, in such a mixture, the content ratio (PDI, 6-HDI or mixtures thereof)/(DDI) is of between 0/100 and 100/0, advantageously of between 25/75 and 0/100, more advantageously between 50/50 and 0/100, in particular 50/50, 25/75, 0,1/99,9 or 0/100. Advantageously, in such a mixture, the content ratio (PDI, 6-HDI or mixtures thereof)/(DDI) is of between 0,1/99,9 and 99,9/0,1, preferably between 20/80 and 80/20 or preferably between 25/75 and 0,1/99,9 or between 50/50 and 0,1/99,9. Typically, the second diisocyanate is hexamethylene diisocyanate (6-HDI), and the content ratio (6-HDI)/(DDI) is of between 0/100 and 100/0, advantageously of between 25/75 and 0/100, more advantageously between 50/50 and 0/100, in particular 50/50, 25/75, 0,1/99,9 or 0/100. Advantageously, the second diisocyanate is hexamethylene diisocyanate (6-HDI), and the content ratio (6-HDI)/(DDI) is of between 0,1/99,9 and 99,9/0,1, preferably between 20/80 and 80/20 or preferably between 25/75 and 0,1/99,9 or between 50/50 and 0,1/99,9.

In the context of the invention, the use of a mixture of diisocyanate, and in particular of DDI with a second diisocyanate has the advantages of controlling and tailoring the final properties of the tissue adhesive such as flexibility, mechanical strength, physico-chemical properties, degradability, biocompatibility, haemostatic properties.

Advantageously, the NCO-terminated urethane prepolymer has a NCO:OH molar ratio of between 1 and 3, advantageously 2. Such a NCO:OH molar ratio allows to obtain a viscosity, a molar mass, a curing time and a biocompatibility adapted to the use as tissue adhesive composition. A NCO:OH molar ratio of less than 1 would lead to increased viscosity and a lack of reactivity, and a NCO:OH molar ratio of more than 2 would lead to high cytotoxicity and low biocompatibility because of the presence of free diisocyanate monomers.

Advantageously, the contents of polyol oligomer and diisocyanate are selected so that the above-mentioned NCO:OH molar ratio is obtained. In particular, the NCO-terminated urethane prepolymer is based on one or several polyol oligomers as disclosed above in a content of between 15 and 50 wt %, and one or several diisocyanates as disclosed above in a total content of between 50 and 85 wt %, by weight in relation to the total weight of the NCO-terminated urethane prepolymer.

Advantageously, the NCO-terminated urethane preprolymer has a free —NCO content of between 4 and 18% by weight, in relation to the total weight of the prepolymer. According to the invention, the free —NCO content is determined by an indirect-titration method. A free —NCO content of less than 4% would lead to insufficient adhesive properties, and a free-NCO content of more than 15 would lead to free diisocyanate monomers and a higher cytotoxicity and lower biocompatibility.

Advantageously, the NCO-terminated urethane prepolymer has a viscosity of between 5 and 120 Pa·s, typically of between 5 and 60 Pa·s or between 15 and 120 Pa·s, advantageously between 70 and 100 Pa·s, the viscosity being measured at temperature of 25° C. in the context of the invention the viscosity has been measured using a TA Instrument Discovery Hybrid Rheometer HR-3 equipped with 20 mm parallel plates. The frequency range was from 10⁵ to 10² s⁻¹ and the gap was 500 μm. Such a viscosity is adapted to the use as tissue adhesive composition. A lower viscosity would in fine lead to a composition with high cytotoxicity and thus unsuitable to be used as a tissue adhesive and a higher viscosity would lead to a composition that cannot be injected.

Method for Preparing the oligoPHA-Diol

The second aspect of the invention relates to a method for preparing polyhydroxyalkanoate-diol oligomers (oligoPHA-diol) having a hydroxyl value of more than or equal to 149 mgKOH/g, comprising the following steps:
a) Heating a reactive solvent at a temperature of more than or equal to the melting point of the PHA and below the reactive solvent boiling point, in particular of between 170° C. and 190° C., advantageously of 180° C., under inert gaz flow, the reactive solvent being selected in the group consisting of 1,2 ethylene glycol, 1,3-propanediol, 1,4-butanediol, and mixtures thereof, preferably 1,4-butanediol;
b) Adding dried PHA and stirring;
c) Starting the reaction by adding a catalyst;
d) Allowing to react for a period of between 15 and 240 minutes, advantageously between 100 and 215 minutes, typically between 205 and 215 minutes;
e) Precipitating the mixture obtained after step d) and washing; and
f) Recovering the oligoPHA-diol by distillation at a temperature of between 120° C. and 180° C., advantageously between 150° C. and 180° C., typically between 140° C. and 160° C., under reduced pressure.

Advantageously, the oligoPHA-diol is as disclosed above, in particular in the section named “NCO-terminated urethane prepolymer”.

Such a method allows the transesterification of a polyhydroxyalkanoate (PHA) with a reactive solvent selected in the group consisting of 1,2-ethylene glycol, 1,3-propanediol (PDO), 1,4-butanediol (BDO), or mixture thereof.

The method for preparing the oligoPHA-diol according to the invention uses biobased reactive solvent and allow to reduce the catalyst concentrations, as well as reduced reactions times. Such a method is thus greener than the method known by the one skilled in the art. Such a method also allows to control the molecular weight of oligoPHA-diol and thus to obtain oligoPHA-diol with lower average molecular weight than 750 g/mol, and higher hydroxyl value than 149 mgKOH/g.

In step a) of said method, the reactive solvent is heated at a temperature of more than or equal to the melting point and below the reactive solvent boiling point of the PHA, and advantageously at a temperature lower than the degradation temperature of the PHA. In particular, the reactive solvent is heated at a temperature of between 170 and 190° C., advantageously of 180° C.

In step a), the reactive solvent is advantageously in an amount of between 2000 and 12000 molar equivalents, typically of between 3000 and 12000 molar equivalents.

In step b), the PHA is advantageously in an amount of 1 molar equivalent. Therefore, advantageously, the molar ratio (reactive solvent)/(PHA) is of between 2000 and 12000 molar equivalents, typically of between 3000 and 12000, advantageously between 2000 and 7000 and more advantageously between 5000 and 7000.

Advantageously, in step b), the solution comprising the reactive solvent and the PHA is stirred until complete dissolution of the PHA, i.e. until the solution is cleared of particles apparent to the human eye.

In step c), once the PHA solution is obtained, the temperature is advantageously set to reaction temperature of between 170° C. and 190° C., more advantageously of 180° C. The reaction is then started by adding a proper amount of catalyst. Advantageously, the catalyst is selected from the group consisting of dibutyltin dilaurate (DBTL), Tin(II) 2-ethylhexanoate, p-toluenesulfonic acid, and mixture thereof, in particular DBTL. Advantageously, the content of catalyst is higher than 0,3% by weight, advantageously between 0,3% and 5%, more advantageously between 0,3% and 1%, more advantageously between 0,5% and 0,75% by weight, in relation to the total weight of the PHA. The method of the invention enables a low dose of catalyst to be used.

In step d), the reaction is carried out for a period of between 15 and 240 minutes, advantageously between 100 and 215 minutes, typically between 205 and 215 minutes.

In step e), at the end of the reaction, the reaction mixture was precipitated and washed, advantageously at least three times, with a large volume of solvent, advantageously of petroleum ether, to remove the catalyst. The solvent, advantageously the petroleum ether, is then separated from the oligoPHA-diol. Residual solvent, advantageously residual petroleum ether, is finally eliminated from the mixture, in particular using a centrifuge 5804 (Eppendorf, France) at 2000 rpm for 2 min.

In step f), the oligoPHA-diol are recovered by distillation at a temperature of between 120 and 180° C., advantageously between 150° C. and 180° C., typically 140-160° C., under reduced pressure.

Advantageously, in this method, the PHA is selected from the group consisting of poly-3-hydroxybutyrate (P3HB), poly-4-hydroxybutyrate (P4HB), poly-3-hydroxyvalerate (P3HV), poly-3-hydroxypropionate (P3HP), poly-4-hydroxyvalerate (P4HV), poly-5-hydroxyvalerate (P5HV), and mixtures thereof, advantageously poly-3-hydroxybutyrate (P3HB), poly-4-hydroxybutyrate (P4HB) or mixtures thereof, more advantageously poly-3-hydroxybutyrate (P3HB).

Advantageously, such a method allows to obtained an oligoPHA-diol having a hydroxyl value of less than or equal to 149 mgKOH/g, advantageously between 149 mgKOH/g and 560 mgKOH/g, advantageously of between 149 mgKOH/g and 375 mgKOH/g, more advantageously of between 160 mgKOH/g and 375 mgKOH/g, in particular of between 224 mgKOH/g and 375 mgKOH/g; and thus an OligoPHA-diol having an average molecular weight of less than or equal to 750 g/mol, more advantageously, of between 200 g/mol and 750 g/mol, more advantageously of between 300 g/mol and 750 g/mol, more advantageously of between 300 g/mol and 700 g/mol, and more advantageously of between 300 g/mol and 500 g/mol.

Advantageously, such a method allows to obtain a oligoPHA-diol having a viscosity of between 1 and 31000 Pa·s, more advantageously between 1 and 10000 Pa·s and more advantageously between 1 and 100 Pa·s, at 25° C., in particular using a TA Instrument Discovery Hybrid Rheometer HR-3 equipped with 20 mm parallel plates. The frequency range was in particular from 10⁵ to 10² s⁻¹ and the gap was 500 μm.

### Method for Preparing NCO-Terminated Urethane Prepolymer

A third aspect of the invention relates to a method for preparing the NCO-terminated urethane prepolymer of the invention, comprising the following steps:
a′) Mixing together the diisocyanate with the polyhydroxyalkanoate-diol oligomers (oligoPHA-diol) at a temperature of between 20° C. and 110° C., under stirring, the diisocyanate being selected from the list consisting of 1,4-butanediisocyanate (BDI), hexamethylene diisocyanate (6-HDI), dimeryl diisocyanate (DDI), pentamethylene diisocyanate (PDI), L-lysine diisocyanate (LDI), 1,7-heptamethylene diisocyanate (7-HDI), and mixtures thereof, and the oligoPHA-diol having a hydroxyl value of more than or equal to 149 mgKOH/g and/or being obtained according to the method of the invention for preparing the oligoPHA-diol;
b′) Optionally, when a mixture of DDI with a second diisocyanate is used, adding in step a′) the DDI and in step b′) adding dropwise the second diisocyanate being selected from the list consisting of 1,4-butanediisocyanate (BDI), pentamethylene diisocyanate (PDI), L-lysine diisocyanate (LDI), hexamethylene diisocyanate (6-HDI), 1,7-heptamethylene diisocyanate (7-HDI), and mixtures thereof;
c′) Allowing to react until obtaining a free —NCO content of between 4 and 18% by weight, in relation to the total weight of the prepolymer, measured by determining the free —NCO content by an indirect-titration method.
d′) Recovering the NCO-terminated urethane prepolymer.

The NCO-terminated urethane prepolymer of the invention is as disclosed above, in particular in the section named “NCO-terminated urethane prepolymer”.

The polyhydroxyalkanoate-diol oligomers (oligoPHA-diol) and the method for preparing the oligoPHA-diol are also as disclosed above, in particular in sections named “NCO-terminated urethane prepolymer” and “Method for preparing the oligoPHA-diol”.

Advantageously, step a′) is carried out at a temperature of between 60° C. and 90° C., more advantageously of between 70° C. and 90° C., in particular at 75° C. In the context of the invention, step a′) can also be carried out by heating the diisocyanate and then adding the oligoPHA-diol under stirring.

Advantageously, the contents of diisocyanate and oligoPHA-diol are selected so that a NCO:OH molar ratio of between 1 and 3, advantageously 2, is obtained. In particular, the content of each diisocyanate is advantageously of between 6 and 85 wt %, the total content of diisocyanate(s) being advantageously of between 50 and 85 wt %, and the content of oligoPHA-diol is advantageously of between 15 and 50 wt %.

Step b′) is carried out when a mixture of DDI with a second diisocyanate is used (see section “NCO-terminated urethane prepolymer”). In such an embodiment, the content of each diisocyanate is selected so that the content ratio (BDI, PDI, LDI, 6-HDI, 7-HDI or mixtures thereof)/(DDI) is of between 0/100 and 100/0, advantageously between 25/75 and 0/100, more advantageously between 50/50 and 0/100, in particular 50/50, 25/75, 0,1/99,9 or 0/100. Advantageously, in such an embodiment, the content of each diisocyanate is selected so that the content ratio (BDI, PDI, LDI, 6-HDI, 7-HDI or mixtures thereof)/(DDI) is of between 0,1/99,9 and 99,9/0,1, advantageously between 20/80 and 80/20 or advantageously between 25/75 and 0,1/99,9, more advantageously between 50/50 and 0,1/99,9, in particular 50/50, 25/75 or 0,1/99,9.

Step c′) is carried out until obtaining a free —NCO content of between 4 and 18% by weight, in relation to the total weight of the prepolymer, measured by determining the free —NCO content by an indirect-titration method. Advantageously, step c′) is carried out during a period of between 150 and 180 minutes. Advantageously, step c′) is carried out at a temperature of between 60° C. and 90° C., more advantageously of between 70° C. and 90° C., in particular at 75° C.

The NCO-terminated urethane prepolymer as obtained by the method of the invention is as disclosed above, in particular in the section named “NCO-terminated urethane prepolymer”.

### Hemostatic Tissue Adhesive Composition and Hemostatic Tissue Adhesive

A fourth aspect of the invention is a hemostatic tissue adhesive composition comprising A) an NCO-terminated urethane preprolymer of the invention or as obtained according to the method of the invention, and B) a chain extender.

Advantageously, the general procedure for preparing said hemostatic tissue adhesive composition is based on a classic two-step method. First, NCO-terminated prepolymers are prepared as described in the previous sections of this application. After reaching the right free NCO content (i.e. a free —NCO content of between 4 and 18% by weight, in relation to the total weight of the prepolymer, measured by determining the free —NCO content by an indirect-titration method), the chain extender B) is added.

Advantageously, the NCO:OH molar ratio in the composition is of between 1 et 2, advantageously between 1 and 1,5, preferably between 1 and 1,2.

Advantageously, the contents of the NCO-terminated prepolymers A) and the chain extender B) are selected so that the above recited NCO:OH molar ratio is obtained. In particular, the content of the NCO-terminated prepolymers A) is advantageously between 50 and 96 wt %, more advantageously between 70 and 96 wt %, more advantageously between 85 and 96 wt %, and the content of the chain extender B) is advantageously between 4 and 50 wt %, more advantageously between 4 and 30 wt %, more advantageously between 4 and 15 wt %.

Advantageously, the chain extender B) is selected from the group consisting of N-ethyldiethanolamine, N-butyldiethanolamine, 1,4-butanediol, polyethylene glycol (PEG), polypropylene glycol (PPG), 1-amino-4-butanol and mixtures thereof.

Advantageously, the chain extender B) is selected from the group consisting of N-ethyldiethanolamine, N-butyldiethanolamine, 1,4-butanediol, polyethylene glycol (PEG) with an average molar mass of between 200 and 1000 g·mol⁻¹, polypropylene glycol (PPG) with an average molar mass of between 400 and 1000 g·mol⁻¹, 1-amino-4-butanol and mixtures thereof, more advantageously 1,4-butanediol.

Advantageously, the chain extender B) is selected from the group consisting of N-ethyldiethanolamine, N-butyldiethanolamine, 1,4-butanediol, PEG200 (^{˜}200 g·mol⁻¹), PEG400 (^{˜}400 g·mol⁻¹), PEG600 (^{˜}600 g·mol⁻¹), PPG420 (^{˜}420 g·mol⁻¹), PPG720 (^{˜}720 g·mol⁻¹), PPG1000 (^{˜}1000 g·mol⁻¹), 1-amino-4-butanol and mixtures thereof, more advantageously 1,4-butanediol.

After curing in contact with the living tissues, the hemostatic tissue adhesive composition of the invention will lead to a hemostatic tissue adhesive. The hemostatic tissue adhesive is typically in the form of a thermoplastic polyurethane (TPU).

The invention thus also relates to a hemostatic tissue adhesive obtained by reacting A) the NCO-terminated urethane prepolymer of the invention or as obtained by the method of the invention; and B) a chain extender advantageously selected from the group consisting of N-ethyldiethanolamine, N-butyldiethanolamine, 1,4-butanediol, polyethylene glycol, polypropylene glycol, 1-amino-4-butanol, and mixtures thereof.

Advantageously, the chain extender B) is selected from the group consisting of N-ethyldiethanolamine, N-butyldiethanolamine, 1,4-butanediol, polyethylene glycol (PEG) with an average molar mass of between 200 and 1000 g·mol⁻¹, polypropylene glycol (PPG) with an average molar mass of between 400 and 1000 g·mol⁻¹, 1-amino-4-butanol and mixtures thereof, more advantageously 1,4-butanediol.

Advantageously, the chain extender B) is selected from the group consisting of N-ethyldiethanolamine, N-butyldiethanolamine, 1,4-butanediol, PEG200 (^{˜}200 g·mol⁻¹), PEG400 (^{˜}400 g·mol⁻¹), PEG600 (^{˜}600 g·mol⁻¹), PPG420 (^{˜}420 g·mol⁻¹), PPG720 (^{˜}720 g·mol⁻¹), PPG1000 (^{˜}1000 g·mol⁻¹), 1-amino-4-butanol and mixtures thereof, more advantageously 1,4-butanediol.

In these aspects, the NCO-terminated urethane prepolymer and its preparation method are as disclosed in the sections above.

In surgery, a fast-curing adhesive is essential to avoid bleeding and/or gas leaks while and after the operation. Therefore, the hemostatic tissue adhesive composition of the invention has a curing time at 25° C. of between 1 and 14000 min, more advantageously of between 1 and 800 min.

### Kit

Another aspect of the invention is a kit for the preparation of a hemostatic tissue adhesive comprising a composition A comprising an NCO-terminated urethane preprolymer of the invention or as obtained according to the method of the invention, and a composition B comprising a chain extender advantageously selected from the group consisting of ethyldiethanolamine, N-butyldiethanolamine, 1,4-butanediol, polyethylene glycol, polypropylene glycol, 1-amino-4-butanol, and mixtures thereof, the compositions A and B being packaged separately and being parenterally administrable simultaneously, sequentially or separately.

Advantageously, the chain extender B) is selected from the group consisting of N-ethyldiethanolamine, N-butyldiethanolamine, 1,4-butanediol, PEG200 (^{˜}200 g·mol⁻¹), PEG400 (^{˜}400 g·mol⁻¹), PEG600 (^{˜}600 g·mol⁻¹), PPG420 (^{˜}420 g·mol⁻¹), PPG720 (^{˜}720 g·mol⁻¹), PPG1000 (^{˜}1000 g·mol⁻¹), 1-amino-4-butanol and mixtures thereof, more advantageously 1,4-butanediol.

In this aspect, the NCO-terminated urethane prepolymer and its preparation method are as disclosed in the sections above.

Advantageously, the kit also comprises an injection means of the compositions A and B, said injection means being advantageously one or more syringes and/or one or more prefillable syringe(s) and/or double chamber syringe and/or one or more catheter(s) or microcatheter(s) for administration of said composition by injection.

### Use

Another aspect of the invention relates to the use of a hemostatic tissue adhesive composition of the invention for the preparation of a hemostatic tissue adhesive. The hemostatic tissue adhesive composition and the hemostatic tissue adhesive being as defined in the above sections.

Another aspect of the invention relates to the use of polyhydroxyalkanoate-diol oligomers (oligoPHA-diol) having a hydroxyl value of more than or equal to 149 mgKOH/g for the preparation of the NCO-terminated urethane prepolymer of the invention. The oligoPHA-diol and the NCO-terminated urethane prepolymer are as defined in the above sections.

### DESCRIPTION OF FIGURES

FIG. 1. represents the OligoPHB-diols Mₙ variation in relation to the reaction time using multiple parameters: a) DBTL molar equivalents (0.7 (rond), 1.5 (triangle), 2.2 (losange), 2.9 (carré)), at T=180° C. and 6000 molar equivalent BDO (FIG. 1A); b) BDO molar equivalents (3000 (rond), 6000 (triangle), 12000 (losange), 18000 (carre)), at T=180° C. and 2.2 molar equivalents DBTL (FIG. 1B); c) temperature (150° C. (rond), 160° C. (triangle), 180° C. (losange)), with 2.2 molar equivalent DBTL and 6000 molar equivalents BDO (FIG. 1C); d) short diol length (ETG (rond), PDO (triangle), BDO (losange)), T=180° C., 2.2 molar equivalent DBTL and 6000 molar equivalents BDO (FIG. 1D).

FIG. 2. represents a)¹H, b)¹³C and c)³¹P NMR spectra of 450 g·mol⁻¹ oligoPHB-diol in CDCl₃.

FIG. 3. represents results of FTIR-ATR analysis of OligoPHB-diols with various Mn. From top to bottom, PHB with decreasing Mn: 260 000 g·mol⁻¹, 1000 g·mol⁻¹, 450 g·mol⁻¹ and 3000 g·mol⁻¹.

FIG. 4. represents ¹H-NMR spectra of a) neat OligoPHB-diol and b) DDI-0% to f) DDI-100% prepolymers, respectively.

FIG. 5. represents the results of FTIR-ATR analysis from top to bottom: neat OligoPHB-diol, DDI-0% to DDI-100% prepolymers, respectively.

FIG. 6. represents the evolution of the free NCO groups content (in %) during a) the reaction between prepolymers with DDI-0%, DDI-25%, DDI-50%, DDI-75% or DDI-100% and BDO and b) DDI-50% prepolymer with different chain extenders (BDA, ABO, BDO, PDO and EtG).

FIG. 7. represents adhesion of the two-component tissue adhesives on muscle tissues for a) all prepolymers (DDI-0%, DDI-25%, DDI-50%, DDI-75% or DDI-100%) with BDO, b) DDI-50% with different chain extenders (BDA, ABO, BDO, PDO and EtG) and c) DDI-50% with BDO on different substrates (Fresh bovine muscle, liver tissue, and porcine skin tissues).

FIG. 8. represents model TPUs tensile properties of a) all prepolymers (DDI-0%, DDI-25%, DDI-50%, DDI-75% or DDI-100%) with BDO and b) DDI-50% with different chain extenders (BDA, ABO, BDO, PDO and EtG).

### EXAMPLES

### Materials.

PHB L88 (Mn=180 000 g/mol, D=2.3 (by SEC)) was kindly supplied by Biocycle, Brazil. IFABOND® was kindly offered by Peters Surgical. 1,4 Butanediol (BDO, 99%) was purchased from Alfa Aesar. Dibutyltin dilaurate (DBTL, 95%), Hexamethylene diisocyanate (HDI, >99%), dibutylamine (>99.5%), bromophenol blue were purchased from Sigma Aldrich. The PHB and the BDO were dried in an oven under vacuum at 40° C. overnight prior to use. Dimeryl diisocyanate (DDI) was kindly supplied by Cognis. Petroleum ether was purchased from VWR.

### General Methods and Analysis.

¹H-NMR spectra in CDCl₃ were implemented with a Bruker 400 MHz spectrophotometer. ¹H-NMR calibration was based on the CDCl₃ chemical shift (δH=7.26 ppm).

Average molar mass (Mn), average mass molar mass (Mₙ) and the polydispersity (D) were measured by Size Exclusion Chromatography (SEC) using an Acquity APC apparatus from Waters in THE (0.6 mL/min) at 40° C. Three columns (Acquity APC XT 450 Å 2.5 lm 4.69150 mm, 200 and 45) were connected. The calibration was performed using PS standards.

The hydroxyl index (IOH) was obtained following DIN 53240-2: 2007-11 (2007), or ASTM E1899-02 (2002).

The free NCO content was obtained by an indirect titration method using DIN 53185, 16945 (1994) or ASTM D1638 (1985)

Shear viscosity of the oligoPHA-diol and of the prepolymers was measured at 25° C. using a TA Instrument Discovery Hybrid Rheometer HR-3 equipped with 20 mm parallel plates. The frequency range was from 1×10⁵ to 100s⁻¹ and the gap was 500 μm.

FTIR-ATR was performed on a Nicolet 380 spectrometer equipped with an ATR diamond module. The spectra were collected with 32 scans.

Curing times were determined using FTIR-ATR and performed on a Nicolet 380 spectrometer with an ATR diamond module. Both components (prepolymer-BDO) were stabilized one night at 25° C. and then mixed together for 15 s. A drop of mixture was then placed on the ATR diamond module. Spectra were acquired after 30 s, 2, 4, 6, 8, 10, 15, 20, 30 min and 1, 2, 4 and 24 h. The NCO conversion (curing time) was determined following the NCO end-groups peak disappearance at 2255 cm⁻¹. The remaining free NCO content was then calculated using the NCO peak area at (A(t)) relative to the NCO peak area at t=0 (A(0)), according to Equation 2:

% NCO(t)=A(t)/A(0)×100 (2)

Adhesion strength in contact with fresh bovine muscle was evaluated by lap shear test, in analogy to the revised ASTM F2255-05 (2005). Strips of tissues of approximately 20×10×3 mm were prepared. Approximatively, 0.2 g of adhesive mixture (prepolymer mixed with BDO) were spread on around 100 mm² tissue surfaces with a spatula, and two pieces of treated strips were then put in contact over the 100 mm², manually pressed together to assure a good contact for few seconds without specific high pressure, to assure inter-adhesion. These specimens were kept at room temperature in an oven with 100% of relative humidity (RH) for 24 h in order to allow curing of the adhesive mixture to the tissue. The evaluations of the adhesion were conducted on a TA Instrument Discovery Hybrid Rheometer HR-3 equipped with the film tension accessory in tensile mode. The crosshead speed was of 5 mm/min. IFABOND® was used as a reference. For the preparation of IFABOND-based specimens, few drops of glues were directly applied on tissue surfaces and two treated strips were pressed together in analogy with the two-component adhesive mixture. Reference tests were performed within the hour after the sample preparation. For each tested adhesive mixture and IFABOND®, sets of minimum five experiments were conducted. The adhesive bond strength (Rmax, in Pa) was obtained by dividing the maximum shear force before failure (in Newtons) by the adhered area (in m²). Results are presented as the mean value with standard deviation.

Uniaxial tensile tests were carried out on the TPU materials using an Instron 5567H (USA) machine equipped with a 10 kN load cell. Experiments were measured at room temperature with a constant crosshead speed of 20 mm·min⁻¹. Sets of five dumbbell-shaped samples with dimensions of approximately 45×5×1 mm³ were tested. Average Young's modulus, tensile strength at break (σₘₐₓ) and elongation at break (εₘₐₓ) were finally determined.

### Example 1: PHB-Diol Oligomers (oligoPHB-Diol) Synthesis According to the Invention and Characterization

Short oligoPHB-diol were synthesized at 180° C. by transesterification reaction of high molar mass PHB using biobased reactive solvent, mostly BDO, with DBTL as catalyst. Reduced catalyst concentrations were used compared to previous studies in order to work in a greener way. Transesterification reaction is described in Scheme 1 and parameters with graphs. Obtained chemical structures were checked and confirmed by ¹H NMR and FTIR. The molar masses, D were determined by SEC in THF.

### Example of Synthesis

1,4 Butanediol (1,4-BDO) (6000 molar equivalents) was heated to 180° C. under argon flow and magnetically stirred. Then, dried PHB (1 molar equivalent) was added and the mixture was stirred until complete PHB complete dissolution. The reaction was started by adding the catalyst, DBTL (2.2 molar equivalent) at 180° C. and the reaction was stopped after 3 h30 by cooling. Precipitation with petroleum ether and several washings with large volumes of petroleum ether were carrying out to remove the catalyst. The trapped petroleum ether was then separated from the mixture using a centrifuge 5804 from Eppendorf, France. Finally, PHB-diol oligomers of 300 g·mol-1 were recovered by 1,4 BDO distillation at 160° C. under reduced pressure. PHB-diol oligomers were dried under vacuum in an oven at 40° C. for at least 12 h before use.

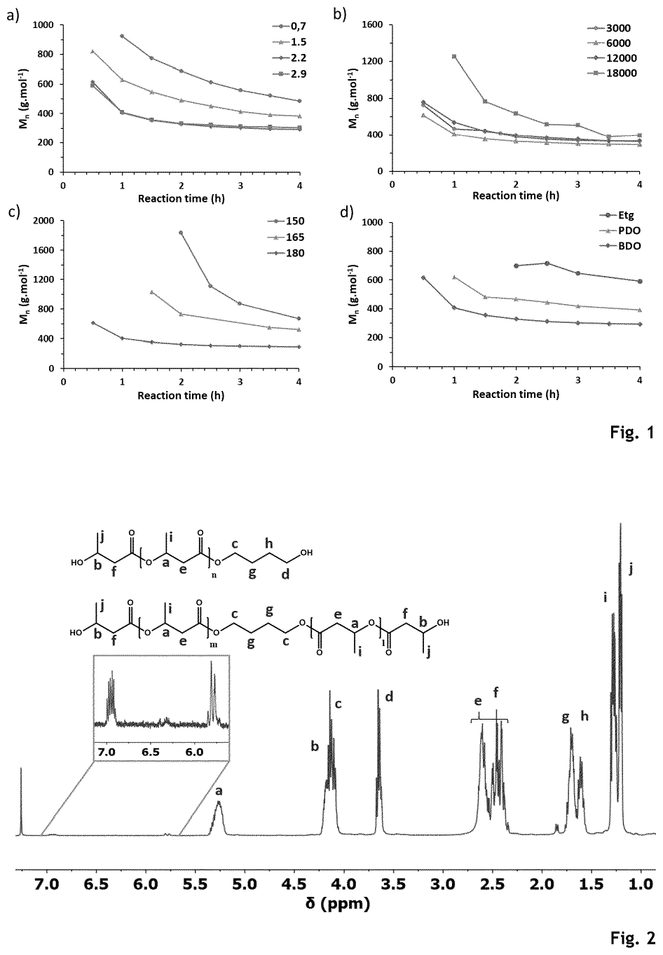

For other Mₙ using multiple parameters, reaction times are referred in the graphic from FIG. 1. As it can be seen, oligoPHB-diols Mₙ decrease over time with a) DBTL molar equivalents (T=180° C. and 6000 molar equivalent BDO) (FIG. 1A), b) BDO molar equivalents (T=180° C. and 2.2 molar equivalents DBTL) (FIG. 1B), c) temperature (with 2.2 molar equivalent DBTL and 6000 molar equivalents BDO) (FIG. 1C), d) short diol length (T=180° C., 2.2 molar equivalent DBTL and 6000 molar equivalents BDO) (FIG. 1D).

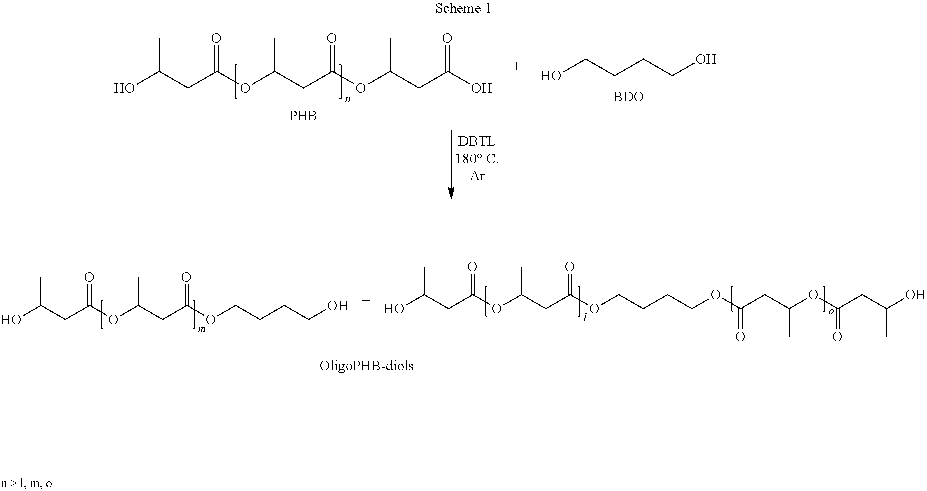

### Chemical and Physico-Chemical Properties Analysis

### ¹H NMR Analysis

Characteristic ¹H-NMR signals for hydroxybutyrate (HB) units were observed at δ=5.29, 2.44-2.65, 1.29 ppm for —CH(CH₃)—CH₂—CO—, —CH(CH₃)—CH₂—CO— and —CH(CH₃)—CH₂—CO— protons respectively. Peaks that correspond to oligoPHB-diol primary hydroxyl end-groups were ascribed at δ=3.67 for HO—CH₂—CH₂-protons and secondary hydroxyl end-groups at δ=4.19 ppm for HO—CH(CH₃)—CH₂— protons. Small peaks at chemical shifts δ=5.81 and 6.96 were assigned to the presence of vinyl end-groups due to PHB thermal degradation and formation of crotonyl end-groups and can be seen zoomed in the box in FIG. 2.

### FTIR-ATR Analysis

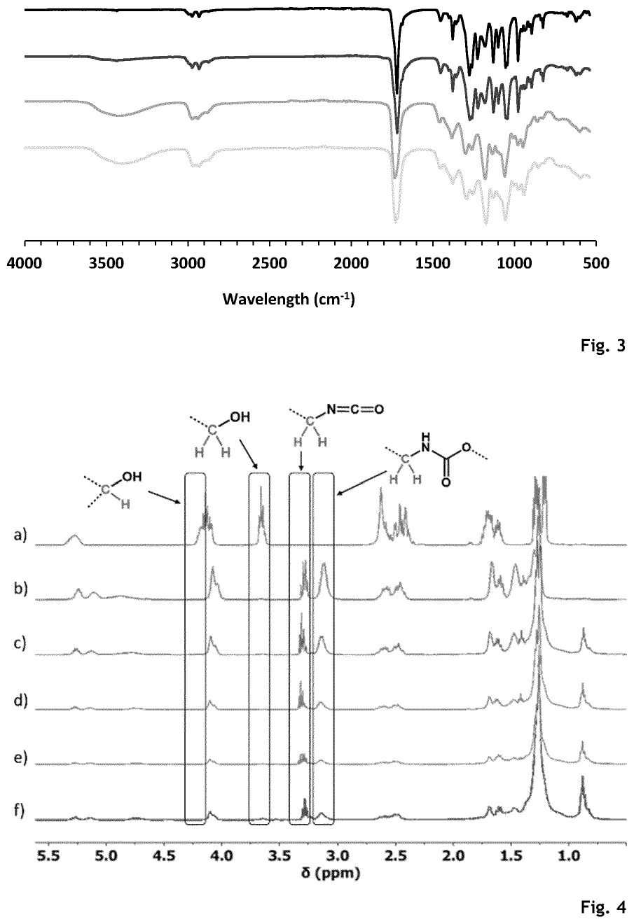

FIG. 3 represents the OligoPHB-diols with various Mₙ: from top to bottom, PHB with decreasing Mₙ: 260 000 g·mol⁻¹, 1000 g·mol⁻¹, 450 g·mol⁻¹ and 3000 g·mol⁻¹. Characteristic peaks from oligoPHB-diol structure were ascribed at 3400, 1720, 1450, 1376, 1172 and 1054 cm⁻¹ corresponding to O—H intermolecular stretching vibration, C═O from ester groups stretching vibration, methyl C—H bending vibration, O—H bending vibration, C—O from ester groups stretching and terminal hydroxyl C—O stretching vibration superimposed with C—O—C stretching from ester, respectively. Small peak intensities of cis-disubstituted C≡C characteristic stretching vibration and its corresponding C—H bending vibration were also found at 1655 cm⁻¹ and 735 cm⁻¹, respectively.

### Molar Masses, IOH and Viscosity Analyses

TABLE 1
Results of molar masses, IOH and viscosity analyses

| | | |
|---|---|---|
| PHB diol Mₙ | Viscosity (25° C.) | |
| (by SEC THF) | [Pa · s] | I_{OH} [mgKOH/g] |

| | | | |
|---|---|---|---|
| ~260000 | g · mol⁻¹ | — | — |
| 1000 | g · mol⁻¹ | — | 110 |
| 700 | g · mol⁻¹ | 30640 ± 1261 | 160 |
| 450 | g · mol⁻¹ | 1.62 ± 0 | 365 |
| 300 | g · mol⁻¹ | 1.60 ± 0 | 420 |

From 1000 g·mol⁻¹ to high Mₙ, the obtained oligoPHB-diols are in form of a solid and thus cannot be processed into a prepolymer without solvent in the second step of reaction (see Debuissy, Pollet, and Averous, 2017).

### Example 2: Prepolymers Synthesis According to the Invention (oligoPHB-Diol300+DDI/HDI) and Characterization

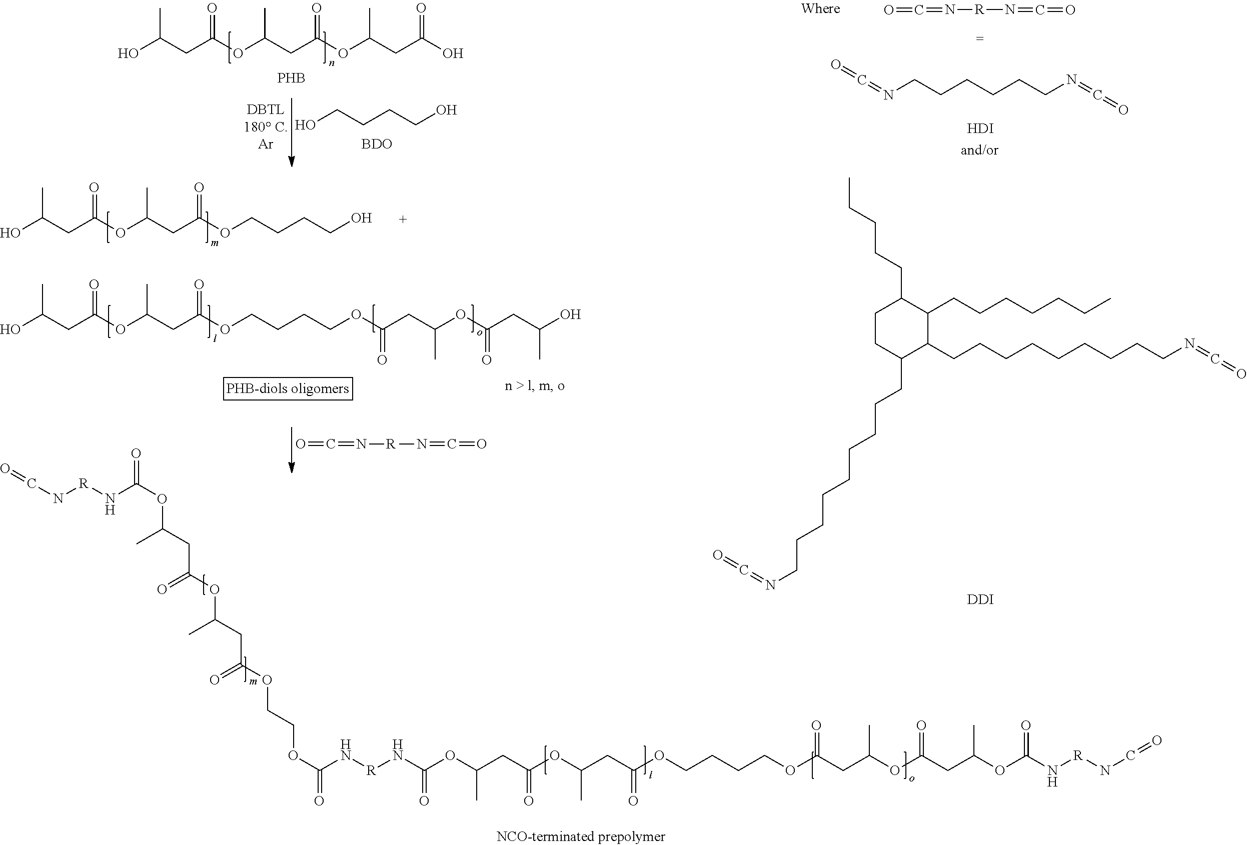

### Synthesis

A series of prepolymers was prepared in a one-step bulk process without catalyst in a green approach. For that, in a previously flame dried three-necked bottom flask equipped with argon flow, a precise amount of DDI was added (see table 2). The reaction was set to 75° C. with an oil bath under mechanical stirring. Previously dried oligoPHB-diol (PHB300 as prepared in example 1) was then added, followed by HDI, dropwise. In the case of neat HDI-based prepolymer, HDI was first added in the three-necked bottom flask, followed by the oligoPHB-diol. The reaction proceeded at 75° C., and the extend was monitored by free NCO content (% NCO) for 2-3 h to yield prepolymers with an NCO/OH molar ration of 2. Five different formulations were prepared and stored at room temperature under argon. They were named DDI-X % according to the mol % of DDI in the diisocyanate content, hence with X varying from 0 to 100 (Table 2).

TABLE 2
Prepolymers formulation (mol % are given in brackets) (Wt
% in relation to the total weight of the prepolymer)

| | PHB300 content | HDI content | DDI content |
|---|---|---|---|
| Prepolymer | [wt %] | [wt %] | [wt %] |
| DDI-0% | 44 | 56 (100) | 0 (0) |
| DDI-25% | 30 | 28 (75) | 42 (25) |
| DDI-50% | 23 | 14 (50) | 63 (50) |
| DDI-75% | 18 | 6 (25) | 76 (75) |
| DDI-100% | 15 | 0 (0) | 85 (100) |

### Chemical and Physico-Chemical Properties Analysis

### NCO Content Determination

The free —NCO content was obtained by an indirect-titration method. For that, DBA (50 mL of a 0.2M solution in dry THF) was added to a known mass of prepolymer (1-2 g) and reacted for 2 min. The resulting amine excess was then back titrated using a standard aqueous HCl 0.5M solution and bromophenol blue as indicator. The NCO content, given in weight percent, was calculated as follows:

$$\% NCO = \frac{( Vb - {V}_{S} ) \times 4202 \times 0.5}{M \times 1000}$$

Where Vb (mL) is the HCl solution volume necessary for the blank titration, Vs (mL) the HCl solution volume required for the sample titration, and M (g) the prepolymer weight.

### 1H NMR

FIG. 4. represents ¹H-NMR spectra of a) neat OligoPHB-diol and b) DDI-0% to f) DDI-100% prepolymers, respectively. Signals at chemical shifts δ=3.67 and 4.19 ppm from oligoPHB-diol protons next to primary and secondary OH respectively disappeared due to the reaction between OH and NCO groups to form urethane bonds. Chemical shift at δ=3.14 ppm was assigned to —CH₂—NH—CO—O protons from HDI and/or DDI and δ=3.29 ppm to protons adjacent to the terminal NCO groups. Characteristic signals of PHB repetitive units at δ=5.29 and 2.44-2.65 ppm were attributed to —CH(CH₃)—CH₂—CO— and —CH(CH₃)—CH₂—CO— protons, respectively, while peaks in the region δ=1.57-1.77 ppm corresponded to the superimposition of —CH₂— protons from all components, thus from BDO, oligoPHB-diol, HDI and DDI chains.

### FTIR-ATR Analysis

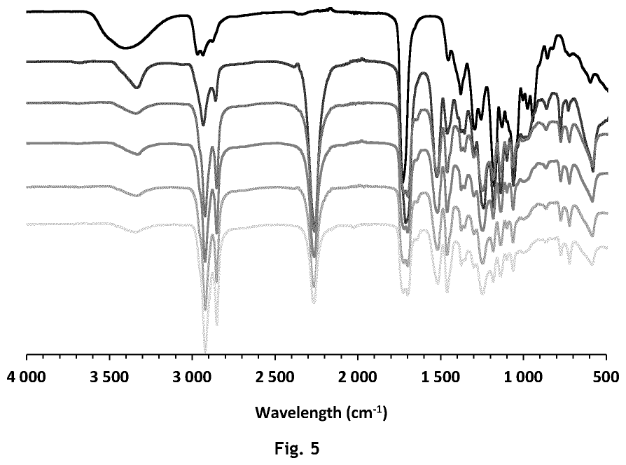

FIG. 5. shows the results of FTIR-ATR analysis from top to bottom: neat OligoPHB-diol, DDI-0% to DDI-100% prepolymers, respectively.

FTIR-ATR analysis was used to confirm the successful synthesis of NCO terminated prepolymers from the oligoPHB-diol. FTIR spectra of oligoPHB-diol and the five prepolymers are depicted in FIG. 5. Polyaddition between oligoPHB-diol and HDI/DDI was demonstrated by the disappearance of the broad O—H stretching vibration from oligoPHB-diol at 3400 cm⁻¹, along with N—H stretching band appearance at 3329 cm⁻¹ from urethane bonds. Other characteristic bands from NCO-terminated polyurethane prepolymer architecture were assigned at 2260, 1700 and 1515 cm⁻¹. They were attributed to N═C═O stretching vibration from prepolymer terminal NCO functions, C═O stretching band from urethane and N—H bending vibration from urethane, respectively.

As DDI content increased in the prepolymer formulation (from DDI-0% to DDI-100%), CH₃ and CH₂ asymmetric stretching vibrations at 2920 and 2851 cm⁻¹, as well as CH_{z} bending vibrations at 1460 cm⁻¹, became stronger due to numerous CH₂ from DDI long aliphatic chains. This observation was in accordance with the Mₙ increase exhibited in Table 3. Moreover, with increasing DDI content there was a gradual decline of the terminal NCO band at 2260 cm⁻¹, when compared to the neat HDI-based prepolymer (DDI-0%). This result is in accordance with the decreasing free % NCO shown in Table 3. Globally, all characteristic urethane peak areas intensities diminished with the addition of DDI in the prepolymer. They decreased to the point where C═O stretching bands from oligoPHB-diol and urethane at 1725 cm⁻¹ and 1700 cm⁻¹, respectively, were clearly distinguishable. On the contrary, for DDI-25% and DDI-0%, these bands were superimposed as a result of higher urethane bond content, in the chains.

### Viscosity Properties

TABLE 3
Prepolymers main physico-chemical properties

| Viscosity at 25° C. was | Mₙ | M_{w} | | Viscosity | Free % NCO |
|---|---|---|---|---|---|
| carried out for the | [g · mol⁻¹] | [g · mol⁻¹] | Ð | [Pa · s] | [%] |
| DDI-0% | 1577 | 2517 | 1.60 | 17 ± 2 | 14.0 |
| DDI-25% | 2543 | 5924 | 2.33 | 47 ± 1 | 9.5 |
| DDI-50% | 3615 | 10548 | 2.92 | 69 ± 6 | 7.1 |
| DDI-75% | 4102 | 7976 | 1.94 | 83 ± 2 | 5.7 |
| DDI-100% | 3959 | 7179 | 1.81 | 101 ± 0 | 4.8 |

### Example 3: Hemostatic Tissue Adhesive Synthesis According to the Invention (PHB-Diol Oligomers 300+DDI/HDI)

The tissue adhesives are composed of two-component systems comprising a prepolymer and BDO. This latter acts as a reactive solvent to maintain a low viscosity for the deliverance of the mix, and at the end to increase the final PU molar mass as chain extender, as for a conventional TPU synthesis (=Hemostatic tissue adhesive).

### Characterization

### Curing Time

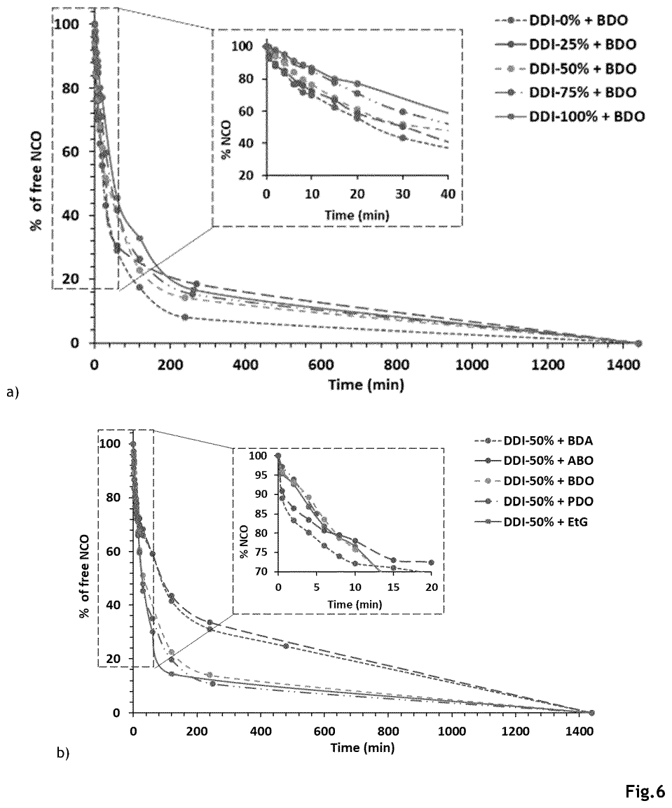

In order to test the reactivity of the NCO-terminated prepolymers, the curing times of the two-component systems were evaluated, since a fast-curing time is essential for surgical adhesives. The corresponding kinetic curves are displayed in FIG. 6a). A significantly higher kinetic, more specifically in the first 40 min, was associated to an increasing HDI content in the prepolymer. This observation is due to multiple phenomena: (i) an increasing oligoPHB-diol content from DDI-100% to DDI-0%, and hence of residual catalyst content. (ii) The initial prepolymers % NCO: It was indeed previously demonstrated that an increased initial % NCO reduces the prepolymers curing time(Gogoi, Alam, and Khandal 2014). Hence, increasing the HDI content results in higher kinetics due to higher initial % NCO (Table 3). Finally, the increase in reactivity with the HDI content can be explained by (iii) the DDI structure influence on the prepolymers: DDI presents a high hydrophobicity due to the long and flexible aliphatic grafted chains (Scheme 2), and its increasing content results in higher prepolymers Mₙ and viscosities (Table 3). In this case, without an initial high mixing, hydrophilic BDO and high content DDI prepolymers can undergo phase segregation due to the difference of hydrophobic/hydrophilic balance, associated with the gap of viscosity. With different chain extenders, due to the higher reactivity of —NH₂ compared to —OH functions and water, in the first 6 min of reaction for ABO and 13 min for BDA, the decrease in free —NCO was more drastic than for the OH bifunctional chain extenders (FIG. 6 b)) in the enlarged square). However, after these times the tendency reversed and the remaining % of free —NCO functions with BDO, PDO and Etg was lower than ABO and BDA for a same reaction time. At 2 h for instance, the free % NCO was around 43% for ABO and BDA, and around 25% for BDO, PDO and Etg.

### Adhesion Strength on Muscle Tissue

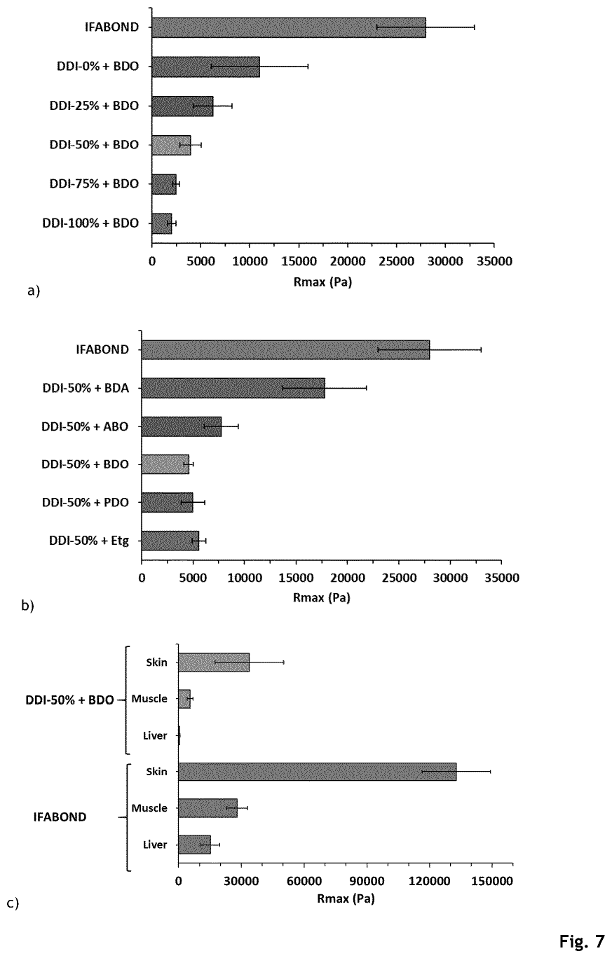

In connection with the targeted biomedical application, adhesives properties of the different systems were further evaluated. The adhesion strength was obtained using a lap-shear test in analogy with ASTM F2255-03 standard. Fresh bovine muscle and liver tissue, as well as porcine skin tissues were used as substrates to test all formulations. Results are depicted in FIG. 7 and display the adhesion strength values (Rₘₐₓ in Pa) for all formulations compared to a well-known commercial reference, IFABOND. Compared to some results obtained in previous studies (McDermott et al. 2004) and to IFABOND, values for the different prepolymers with BDO are rather low, ranging from 2 to 11 kPa. In our two-component systems, increasing HDI content resulted in an improved adhesion strength, most probably due to a higher urethane bonds density and thus physical interactions with the tissues after curing. Moreover, lower initial % NCO in DDI-based systems (Table 3) drops the global adhesion strength capacity of the adhesive. Varying the chain extender using high reactivity functional groups such as NH₂ significantly improved the adhesion on muscle tissues: the average adhesion strength gradually increased from 4590 to 17770 Pa for BDO and BDA, respectively. Reducing the chain extender chain length also increased adhesion but to a much lower extend (up to 5600 Pa for Etg).

### TPUs Model Polymers Analysis

### Synthesis

A series of five TPUs (hemostatic tissue adhesive) was prepared from PHB-diol oligomers with HDI and/or DDI as diisocyanates and BDO as chain extender. The general procedure for the TPU preparation was based on a classic two-step method. First, NCO-terminated prepolymers were prepared by adding oligoPHB-diol to different proportions of HDI and DDI with a NCO:OH molar ratio of 2, as described in Example 2. After determination of the NCO content, an exact amount of chain extender was added (See Table 4). The reaction was then vigorously stirred for one minute and subsequently poured in a Teflon mold. Preparations were kept overnight in an oven at 80° C. to ensure completion of the reaction. Finally, TPUs were compression-molded in a hot press at 120° C. with 200 MPa pressure for 5 min, followed by 10 min quenching between two steel-plates to obtain 1 mm thickness films.

TABLE 4
TPUs-based Hemostatic adhesives compositions

| | Prepolymer | | | | |
|---|---|---|---|---|---|
| | OligoPHB- | | DDI | | Chain extender |
| | diol content | HDI content | content | Chain | content |
| TPU | [wt %] | [wt %] | [wt %] | extender | [wt %] |
| DDI-0%-BDO | 39 (1) | 49 (2) | 0 (0) | BDO | 12 (1) |
| DDI-25%-BDO | 27 (1) | 26 (1.5) | 38 (0.5) | BDO | 9 (1) |
| DDI-50%-BDO | 21 (1) | 13 (1) | 59 (1) | BDO | 7 (1) |
| DDI-75%-BDO | 17 (1) | 5 (0.5) | 71 (1.5) | BDO | 7 (1) |
| DDI-100%-BDO | 14 (1) | 0 (0) | 81 (2) | BDO | 5 (1) |
| DDI-50%-BDA | 21 (1) | 13 (1) | 59 (1) | BDA | 7 (1) |
| DDI-50%-ABO | 21 (1) | 13 (1) | 59 (1) | ABO | 7 (1) |
| DDI-50%-PDO | 21 (1) | 14 (1) | 61 (1) | PDO | 4 (1) |
| DDI-50%-Etg | 21 (1) | 14 (1) | 61 (1) | Etg | 4 (1) |

### Mechanical Properties

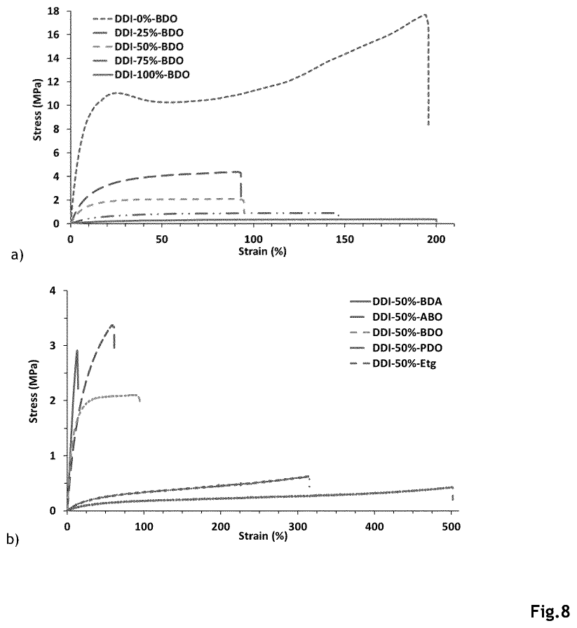

Uniaxial Stress-strain curves at room temperature are shown in FIG. 8 in order to evaluate the mechanical behavior of the TPU and to mimic a potential stress solicitation of the final materials in situ. The corresponding values are shown in Table 5. Increasing the HDI content resulted to a significant increase of the elastic modulus and tensile strength, up to a Young's modulus of 174 MPa for DDI-0%. DDI-0%-BDO exhibited a typical thermoplastic polymer behavior with plastic deformation region after the yield point, followed by an increasing stress until rupture. The other materials containing increasing DDI contents had higher elongations and lower tensile strengths at break, which are conventional behaviors of thermoplastic elastomers (TPE), as it is usually the case for most TPUs. Elongation at break increased with the DDI fraction, which bring mobility and softness with decreasing Young's modulus, as expected for this type of material. TPU-urea PHB300-ABO and PHB300-BDA are hard and brittle polymers with high Young's Modulus and short elongations at break, compared to PHB300-BDO. It is common for PU-urea to achieve high tensile strength due to greater physical crosslink from N—H bonds in urea, which correlates to high Tg(HS) described by DSC earlier. With regard to the chain extender length, harder polymers were obtained by increasing the number of carbons in the chain. In fact, average Young's moduli from 0.6 to 24.4 MPa were obtained for PHB300-Etg and PHB300-BDO, respectively. Usually, increasing the chain length leads to more flexible polymers, but this tendency is reversed for very short chains. Here, it was clear that the incorporation of shorter diols (2 and 3 carbons) resulted in more flexible chains with a gradual increase of the elongation at break (up to 517% with Etg).

TABLE 5
Mechanical properties of the model TPUs

| | Elastic modulus | Tensile strength at | Elongation at |
|---|---|---|---|
| TPU | [MPa] | break [MPa] | break [%] |
| DDI-0%-BDO | 174.0 ± 1.3 | 11.2 ± 0.5 | 194 ± 14 |
| DDI-25%-BDO | 39.0 ± 1.0 | 4.4 ± 0.1 | 97 ± 6 |
| DDI-50%-BDO | 24.4 ± 0.3 | 2.1 ± 0.0 | 96 ± 1 |
| DDI-75%-BDO | 5.5 ± 0.1 | 0.9 ± 0.0 | 147 ± 5 |
| DDI-100%-BDO | 1.6 ± 0.1 | 0.5 ± 0.0 | 185 ± 35 |
| DDI-50%-BDA | 27.3 ± 1.5 | 2.8 ± 0.1 | 13 ± 1 |
| DDI-50%-ABO | 17.7 ± 0.4 | 3.6 ± 0.1 | 72 ± 11 |
| DDI-50%-PDO | 1.1 ± 0.0 | 0.7 ± 0.1 | 336 ± 20 |
| DDI-50%-Etg | 0.6 ± 0.2 | 0.4 ± 0.0 | 517 ± 12 |

1. A NCO-terminated urethane preprolymer based on at least a polyol oligomer and a diisocyanate, wherein the polyol oligomer is polyhydroxyalkanoate-diol oligomer (oligoPHA-diol) having a hydroxyl value of more than or equal to 149 mgKOH/g, and the diisocyanate is selected from the list consisting of 1,4-butane diisocyanate (BDI), hexamethylene diisocyanate (6-HDI), dimeryl diisocyanate (DDI), pentamethylene diisocyanate (PDI), L-lysine diisocyanate (LDI), 1,7-heptamethylene diisocyanate (7-HDI), and mixtures thereof.

2. The NCO-terminated urethane preprolymer of claim 1, wherein the oligoPHA-diol are obtained by transesterification of a polyhydroxyalkanoate (PHA) with a reactive solvent selected in the group consisting of 1,2-ethylene glycol, 1,3-propanediol (PDO), 1,4-butanediol (BDO), or mixture thereof.

3. The NCO-terminated urethane preprolymer of claim 1 or 2, wherein the oligoPHA-diol have a hydroxyl value of between 149 mgKOH/g and 560 mgKOH/g, advantageously of between 149 mgKOH/g and 375 mgKOH/g, more advantageously of between 160 mgKOH/g and 375 mgKOH/g, in particular of between 224 mgKOH/g and 375 mgKOH/g.

4. The NCO-terminated urethane preprolymer of any one of claims 1 to 3, wherein the PHA is selected from the group consisting of poly-3-hydroxybutyrate (P3HB), poly-4-hydroxybutyrate (P4HB), poly-3-hydroxyvalerate (P3HV), poly-3-hydroxypropionate (P3HP), poly-4-hydroxyvalerate (P4HV), poly-5-hydroxyvalerate (P5HV), and mixtures thereof, advantageously poly-3-hydroxybutyrate (P3HB), poly-4-hydroxybutyrate (P4HB) or mixtures thereof, more advantageously poly-3-hydroxybutyrate (P3HB).

5. The NCO-terminated urethane preprolymer of any one of claims 1 to 4, wherein the diisocyanate is a mixture of dimeryl diisocyanate (DDI) with a second diisocyanate selected in the group consisting of 1,4-butanediisocyanate (BDI), pentamethylene diisocyanate (PDI), L-lysine diisocyanate (LDI), hexamethylene diisocyanate (6-HDI), 1,7-heptamethylene diisocyanate (7-HDI), and mixtures thereof, in a content ratio (BDI, PDI, LDI, 6-HDI, 7-HDI or mixtures thereof)/(DDI) of between 0,1/99,9 and 99,9/0,1, advantageously between 20/80 and 80/20 or advantageously between 25/75 and 0,1/99,9.

6. The NCO-terminated urethane preprolymer of claim 5, wherein the second diisocyanate is pentamethylene diisocyanate (PDI), hexamethylene diisocyanate (6-HDI), or mixtures thereof, advantageously hexamethylene diisocyanate (6-HDI).

7. The NCO-terminated urethane preprolymer of any one of claims 1 to 6, wherein the NCO-terminated urethane prepolymer has a NCO:OH molar ratio of between 1 and 3, advantageously 2.

8. The NCO-terminated urethane preprolymer of any one of claims 1 to 7, wherein the NCO-terminated urethane preprolymer has a free —NCO content of between 4 and 15% by weight, in relation to the total weight of the prepolymer.

9. The NCO-terminated urethane preprolymer of any one of claims 1 to 8, wherein the NCO-terminated urethane preprolymer has a viscosity of between 5 and 120 Pa·s, advantageously between 5 and 60 or advantageously between 70 and 100 Pa·s, the viscosity being measured at temperature at a temperature of 25° C. by shear viscosity between parallel plates.

10. A method for preparing polyhydroxyalkanoate-diol oligomers (oligoPHA-diol) having a hydroxyl value of more than or equal to 149 mgKOH/g, comprising the following steps:
a) Heating a reactive solvent at a temperature of more than or equal to the melting point of the PHA and below the reactive solvent boiling point, in particular of between 170° C. and 190° C., advantageously of 180° C., under inert gaz flow, the reactive solvent being selected in the group consisting of 1,2 ethylene glycol, 1,3-propanediol, 1,4-butanediol, and mixtures thereof, preferably 1,4-butanediol;
b) Adding dried PHA and stirring;
c) Starting the reaction by adding a catalyst;
d) Allowing to react for a period of between 15 and 240 minutes, advantageously between 100 and 215 minutes, typically between 205 and 215 minutes;
e) Precipitating the mixture obtained after step d) and washing; and
f) Recovering the oligoPHA-diol by distillation at a temperature of between 120° C. and 180° C., advantageously between 150° C. and 180° C., typically between 140° C. and 160° C., under reduced pressure.

11. The method of claim 10, wherein the molar ratio (reactive solvent)/(PHA) is of between 2000 and 12000, advantageously between 2000 and 7000 and more advantageously between 5000 and 7000.

12. The method of claim 10 or 11, wherein the PHA is selected from the group consisting of poly-3-hydroxybutyrate (P3HB), poly-4-hydroxybutyrate (P4HB), poly-3-hydroxyvalerate (P3HV), poly-3-hydroxypropionate (P3HP), poly-4-hydroxyvalerate (P4HV), poly-5-hydroxyvalerate (P5HV), and mixtures thereof, advantageously poly-3-hydroxybutyrate (P3HB), poly-4-hydroxybutyrate (P4HB) or mixtures thereof, more advantageously poly-3-hydroxybutyrate (P3HB).

13. A method for preparing NCO-terminated urethane prepolymer as defined in any one of claims 1 to 9, comprising the following steps:
a′) Mixing together the diisocyanate with the polyhydroxyalkanoate-diol oligomer (oligoPHA-diol) at a temperature of between 20° C. and 110° C., under stirring, the diisocyanate being selected from the list consisting of 1,4-butanediisocyanate (BDI), hexamethylene diisocyanate (6-HDI), dimeryl diisocyanate (DDI), pentamethylene diisocyanate (PDI), L-lysine diisocyanate (LDI), 1,7-heptamethylene diisocyanate (7-HDI), and mixtures thereof, and the oligoPHA-diol having a hydroxyl value of more than or equal to 149 mgKOH/g and/or being obtained according to the method of any one of claims 10 to 12;
b′) Optionally, when a mixture of DDI with a second diisocyanate is used, adding in step a′) the DDI and in step b′) adding dropwise the second diisocyanate being selected from the list consisting of 1,4-butanediisocyanate (BDI), pentamethylene diisocyanate (PDI), L-lysine diisocyanate (LDI), hexamethylene diisocyanate (6-HDI), 1,7-heptamethylene diisocyanate (7-HDI), and mixtures thereof;
c′) Allowing to react until obtaining a free —NCO content of between 4 and 18% by weight, in relation to the total weight of the prepolymer, measured by determining the free —NCO content by an indirect-titration method.
d′) Recovering the NCO-terminated urethane prepolymer.

14. A hemostatic tissue adhesive composition comprising A) an NCO-terminated urethane preprolymer as defined in any one of claims 1 to 9 or as obtained according to the method of claim 13, and B) a chain extender.

15. The hemostatic tissue adhesive composition of claim 14, wherein the chain extender B) is selected from the group consisting of N-ethyldiethanolamine, N-butyldiethanolamine, 1,4-butanediol, polyethylene glycol, polypropylene glycol, 1-amino-4-butanol and mixtures thereof, advantageously 1,4-butanediol.

16. The hemostatic tissue adhesive composition of claims 14 or 15, wherein the NCO:OH molar ratio in the composition is of between 1 et 2, advantageously between 1 and 1,5, preferably between 1 and 1,2.

17. A hemostatic tissue adhesive obtained by reacting A) the NCO-terminated urethane prepolymer as defined in any one of claims 1 to 9 or as obtained by the method of claim 13; and B) a chain extender advantageously selected from the group consisting of N-ethyldiethanolamine, N-butyldiethanolamine, 1,4-butanediol, polyethylene glycol, polypropylene glycol, 1-amino-4-butanol, and mixtures thereof, more advantageously 1,4-butanediol.

18. A kit for the preparation of a hemostatic tissue adhesive comprising a composition A comprising an NCO-terminated urethane preprolymer as defined in any one of claims 1 to 9 or as obtained according to the method of claim 13, and a composition B comprising a chain extender advantageously selected from the group consisting of ethyldiethanolamine, N-butyldiethanolamine, 1,4-butanediol, polyethylene glycol, polypropylene glycol, 1-amino-4-butanol, and mixtures thereof, more advantageously 1,4-butanediol, the compositions A and B being packaged separately and being parenterally administrable simultaneously, sequentially or separately.

19. Use of a hemostatic tissue adhesive composition as defined in any one of claims 14 to 16 for the preparation of a hemostatic tissue adhesive.
